(19) United States
(12) Patent Application Publication
(10) Pub. No.: US 2025/0049506 A1
(43) Pub. Date: Feb. 13, 2025

(54) PATH DETERMINATION METHOD, ELECTRONIC APPARATUS AND COMPUTER-READABLE STORAGE MEDIUM

(71) Applicant: NOAHTRON INTELLIGENCE MEDTECH (HANGZHOU) CO., LTD., Hangzhou, Zhejiang (CN)

(72) Inventors: Shandeng Huang, Hangzhou, Zhejiang (CN); Lufeng Pan, Hangzhou, Zhejiang (CN); Xiukun Zhang, Hangzhou, Zhejiang (CN); Jianfei Liu, Hangzhou, Zhejiang (CN)

(21) Appl. No.: 18/722,794

(22) PCT Filed: Oct. 26, 2022
(86) PCT No.: PCT/CN2022/127607

§ 371 Date: Jun. 21, 2024

(30) Foreign Application Priority Data
Dec. 21, 2021 (CN) 202111585641.4

(51) Int. Cl. A61B 34/10; A61B 17/00; A61B 17/34; A61B 34/20; A61B 34/30

(52) U.S. Cl. CPC A61B 34/10; A61B 17/3403; A61B 34/20; A61B 34/30; A61B 2017/00398; A61B 2017/3413; A61B 2034/107; A61B 2034/2063; A61B 2034/304

(57) ABSTRACT

A path determination method, an electronic apparatus and a computer-readable storage medium are disclosed, which enable an accurate implementation of puncture surgery, and improve a safety of surgery. The method acquires a first and a second ultrasound section, which are collected by at least one ultrasound probe. An intersecting line of the first and the second ultrasound section is determined in the first ultrasound section and the second ultrasound section respectively. When the first ultrasound section and/or the second ultrasound section meet(s) a preset condition, a puncture travel path is determined based on the intersecting line, wherein the puncture travel path is a travel path along which a puncture needle performs puncturing.

### TECHNICAL FIELD

The present application relates to the field of medical instruments, and particularly to a path determination method, an electronic apparatus, and a computer-readable storage medium.

### DESCRIPTION OF THE PRIOR ART

In the medical field, puncture refers to a diagnostic and treatment technology involving the insertion of a puncture needle into an organ of a biological body to extract secretions, inject gas or medication into a body cavity, extract living tissue from the organ, or perform local ablation of the organ. The most common puncture method used in clinical is guided under two-dimensional ultrasonic image. However, the two-dimensional ultrasound only indicates plane images, while finding a position of the needle tip or even the entire needle has high requirements for operational skills and spatial imagination ability of an operator for the puncture, especially for the organ such as the heart that are constantly in motion, puncture is more difficult. With the development of robotics technology, robots (or mechanical arms) have been applied to puncture procedures. Most of typical puncture robots use pre-operative CT and intraoperative X-ray image to perform image registration or use optical ball positioning registration, to thereby plan puncture paths.

However, both image registration and optical ball positioning registration have errors, especially for organs that move with breathing or heartbeat. Planning the puncture paths through registration methods will cause greater errors. Therefore, for punctures of organs in abdominal and thoracic cavities, clinically can only be manually completed by the surgeon's experience, and there are still operational errors due to various unstable factors.

### SUMMARY OF THE DISCLOSURE

A path determination method, an electronic apparatus, and a computer-readable storage medium provided in an embodiment of the present application can improve an accuracy of the puncture, thereby assisting an accurate implementation of puncture surgery and improving a safety of surgery.

The embodiment of the present application provides a path determination method, which includes:
obtaining a first ultrasound section and a second ultrasound section collected by at least one ultrasound probe;
determining an intersecting line of the first ultrasound section and the second ultrasound section; and
determining a puncture travel path according to the intersecting line, when receiving a stop command triggered based on the first ultrasound section and/or the second ultrasound section.

The embodiment of the present application also provides a path determination device, which includes:
an acquisition module, configured to obtain a first ultrasound section and a second ultrasound section collected by at least one ultrasound probe;
a first determination module, configured to determine an intersecting line of the first ultrasound section and the second ultrasound section; and
a second determination module, configured to determine a puncture travel path according to the intersecting line, when receiving a stop command triggered based on the first ultrasound section and/or the second ultrasound section.

The embodiment of the present application also provides a medical robot, which includes at least one probe mechanical arm, and a puncture mechanical arm. The at least one probe mechanical arm holds an ultrasound probe, and the puncture mechanical arm holds a puncture needle. The medical robot also includes a control component.

The control component is configured to control the ultrasound probe held by the at least one probe mechanical arm to move and obtain a first ultrasound section and a second ultrasound section collected by the ultrasound probe, determine an intersecting line of the first ultrasound section and the second ultrasound section, and determine a puncture travel path according to the intersecting line, when receiving a stop command triggered based on the first ultrasound section and/or the second ultrasound section. The puncture travel path is used to indicate the puncture needle held by the puncture mechanical arm clamp for puncture.

The embodiment of the present application also provides an electronic apparatus, which includes a memory and a processor.

The memory stores executable program codes.

The processor, coupled with the memory, is configured to call the executable program codes stored in the memory, and execute the path determination method provided by the above embodiment.

The embodiment of the present application also provides a non-transitory computer-readable storage medium storing computer programs. When the computer programs are running by a processor, the path determination method provided by the above embodiment is implemented.

From above-mentioned technical schemes provided by the present application, it can be seen that generating the puncture travel path through the intersecting line of a plurality of ultrasound sections can achieve a precise localization of the puncture path, enabling the puncture needle to accurately locate a target point during puncture along the puncture travel path, thereby improving an accuracy of puncture. Additionally, synchronously displaying the puncture travel path on a first ultrasound image and a second ultrasound image, enables a visualization of the puncture needle during a puncture process, accurately locating a position of the puncture needle, and enabling a full-process monitoring of the puncture process, thereby improving the safety of the surgery.

### BRIEF DESCRIPTION OF DRAWINGS

In order to illustrate the embodiments of the present application or the technical solutions in the prior art more clearly, the drawings used in the embodiments or the prior art description will be briefly introduced below. Apparently, the drawings in the following descriptions are merely some embodiments of the present application, and those skilled in the art can obtain other drawings from these drawings without any creative work.

FIG. 1-a shows a schematic flowchart of a path determination method according to an embodiment of the present application.

FIG. 1-b is a schematic view showing a motion platform, and a static coordinate system and a dynamic coordinate system established based on the motion platform according to an embodiment of the present application.

FIG. 2 shows a structural schematic diagram of a path determination device according to an embodiment of the present application.

FIG. 3 shows a structural schematic diagram of a medical robot according to an embodiment of the present application.

FIG. 4 shows a structural schematic diagram of an electronic apparatus according to an embodiment of the present application.

FIG. 5 is a structural schematic view of a medical robot according to another embodiment of the present application.

### DESCRIPTION OF EMBODIMENTS

In order to make the purposes, features, and advantages of the present application be more clearly, the present application will be described in detail below with reference to the drawings and specific embodiments. Apparently, described embodiments are merely embodiments which are a part of the present application, rather than all embodiments. All the other embodiments obtained by those of ordinary skill in the art according to the embodiments of the present application without creative efforts should fall within the scope of the present application.

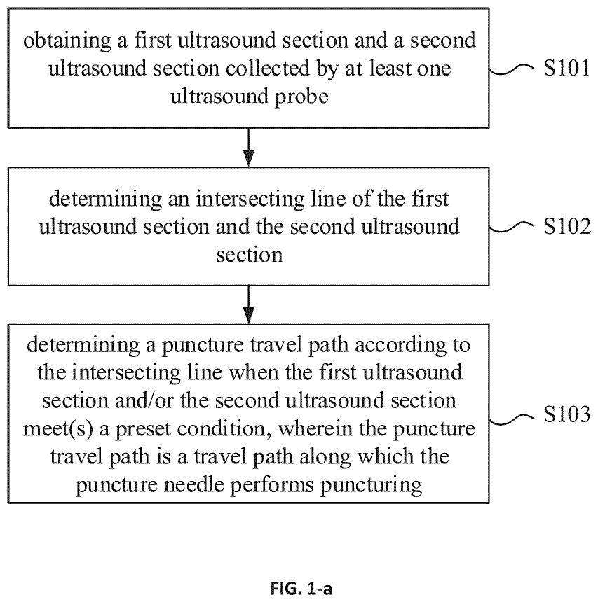

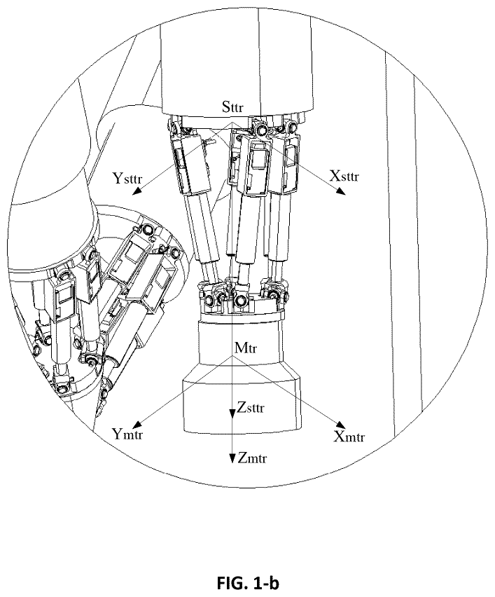

Referring to FIG. 1-a, it shows a flowchart of performing a path determination method according to an embodiment of the present application. The method can be applied to various surgeries using puncture needles or ablation needles. As shown in FIG. 1-a, the method mainly includes steps as follows.

It should be noted that the term “control” mentioned here, which involves controlling the ultrasound probe held by the mechanical arm to move, does not refer to manually control by a surgeon performing the puncture surgery. Instead, the operator performing the puncture surgery only needs to issue commands through a device. After receiving the commands, computer programs will control the ultrasound probe held by the mechanical arm to move.

Step S101, obtaining a first ultrasound section and a second ultrasound section collected by at least one ultrasound probe.

It should be noted that in the embodiments of the present application, the first ultrasound section and the second ultrasound section can be collected by a same ultrasound probe, alternatively, the first ultrasound section and the second ultrasound section can be collected by different ultrasound probes.

For example, the first ultrasound section and the second ultrasound section can be collected by the same ultrasound probe. The same ultrasonic probe collects the first ultrasound section and the second ultrasound section in different positions. That is, the ultrasound probe moves to a first position to collect the first ultrasound section and save the first ultrasound section, which can be sent to a designated storage unit for storage, etc., and then the same ultrasound probe moves to a second position to collect the second ultrasound section. That is, the same ultrasound probe collects the first ultrasound section in the first position and the second ultrasound section in the second position. In order to facilitate the descriptions, the ultrasound probe can be referred to as a first ultrasound probe when collecting the first ultrasound section, and as a second ultrasound probe when collecting the second ultrasound section. Correspondingly, the mechanical arm holding the ultrasound probe is referred to as a first mechanical arm or called as a first position of the mechanical arm, when collecting the first ultrasound section; and as a second mechanical arm or called as a second position of the mechanical arm when collecting the second ultrasound section.

For another example, the first ultrasound section and the second ultrasound section can be collected by different ultrasound probes. For instance, one ultrasound probe (e.g., referred to as a first ultrasound probe) collects the first ultrasound section in a first position, and then another ultrasound probe (e.g., referred to as the second ultrasound probe) collects the second ultrasound section in a second position. Correspondingly, for the mechanical arms holding the two ultrasound probes, the mechanical arm holding the first ultrasound probe is referred to as a first mechanical arm, and the mechanical arm holding the second ultrasound probe is referred to as a second mechanical arm.

In the embodiments below, there is no distinction for the two examples described above. Those skilled in the art will realize that the first ultrasound probe and the second ultrasound probe described in the embodiments below may be the same ultrasound probe or different ultrasound probes. Correspondingly, the first mechanical arm and the second mechanical arm described in the embodiments below may be the same mechanical arm or different mechanical arms. In case that the first mechanical arm and the second mechanical arm are the same mechanical arm, it can also be referred to as the first position of the mechanical arm and the second position of the mechanical arm.

In one embodiment, a first probe coordinate system can be established according to the first ultrasound probe. For example, an end point of the first ultrasound probe can be taken as an origin point of the first probe coordinate system, a direction along the first ultrasound probe as a z-axis direction, accordingly an x-axis direction and a y-axis direction can be determined. For example, in an initial state, the first ultrasound probe may be vertically relative to a surface of a skin of an object, and the z-axis direction is perpendicular to the surface of the skin and points towards the skin.

Similarly, a second probe coordinate system can be established according to the second ultrasound probe. For example, an end point of the second ultrasound probe can be taken as an origin point of the second probe coordinate system, a direction along the second ultrasound probe as a z-axis direction, accordingly an x-axis direction and a y-axis direction can be determined.

In one embodiment, the first ultrasound section can be a plane formed by the x-axis and the z-axis of the first probe coordinate system, and the second ultrasound section can be a plane formed by the x-axis and the z-axis of the second probe coordinate system.

Of course, the above-mentioned method of establishing the coordinate systems is only an illustrative example. In practice, other methods can also be used to establish coordinate systems, and the embodiment is not specifically limited.

Step S102, determining an intersecting line of the first ultrasound section and the second ultrasound section.

According to a principle of Euclidean geometry, as long as two planes are not parallel, the two planes must intersect, and the intersection of planes means that they have an intersecting line. Here, taking a case that the first ultrasound section is collected by the first ultrasound probe, the second ultrasound section is collected by the second ultrasound probe, and the first ultrasound probe and the second ultrasound probe are the same ultrasound probe as an example, an implementation of determining the intersecting line of the first ultrasound section and the second ultrasound section in the first ultrasound section and the second ultrasound section, respectively, can be as follows: first, determine plane equations corresponding to the first ultrasound section and the second ultrasound section in the same probe coordinate system, and then, obtain the intersecting line of the first ultrasound section and the second ultrasound section by solving a plane equation corresponding to the first ultrasound section and a plane equation corresponding to the second ultrasound section in the same probe coordinate system. Wherein, the same probe coordinate system can be the first probe coordinate system or the second probe coordinate system. The first probe coordinate system is established according to the first ultrasound probe. The second probe coordinate system is established according to the second ultrasound probe. It should be noted that although the above description takes the example of the first ultrasound probe and the second ultrasound probe being the same ultrasound probe to illustrate the implementation scheme of determining the intersecting line of the first ultrasound section and the second ultrasound section in the first ultrasound probe and the second ultrasound probe, the first ultrasound probe and the second ultrasound probe may be different probes. The implementation principles of the scheme are the same, and will not be repeated here.

In an embodiment, determining the plane equations corresponding to the first ultrasound section and the second ultrasound section in the same probe coordinate system can involve converting the ultrasound section in one coordinate system to another coordinate system.

Taking the same probe coordinate system as the first probe coordinate system as an example, since the first probe coordinate system is established according to the first ultrasound probe and the first ultrasound section is collected by the first ultrasound probe, the plane equation corresponding to the first ultrasound section in the first probe coordinate system can be calculated. Similarly, the plane equation corresponding to the second ultrasound section in the second probe coordinate system can also be calculated. As for how to obtain the plane equation corresponding to the second ultrasound section in the first probe coordinate system, it can be determined according to a transformation relationship between the first probe coordinate system and the second probe coordinate system. Wherein, the transformation relationship can include a translation transformation and a rotation transformation, which can be specifically determined according to a position relationship between the first ultrasound probe and the second ultrasound probe.

In an embodiment, a mechanical arm can be equipped with a motion platform. The ultrasound probe held by the mechanical arm is controlled according to a parallel structure of the motion platform. The motion platform can include a dynamic platform and a static platform. According to the static platform, a static coordinate system (referred to as a mechanical arm motion platform static coordinate system) can be established. According to the dynamic platform, a dynamic coordinate system (referred to as a mechanical arm motion platform dynamic coordinate system) can be established. As shown in FIG. 1-b, Sₜₜᵣ represents an origin point of the mechanical arm motion platform static coordinate system, X_{Sttr}, Y_{Sttr}, and Z_{Sttr} represent an X-axis, a Y-axis, and a Z-axis of the mechanical arm motion platform static coordinate system, respectively. M, represents an origin point of the mechanical arm motion platform dynamic coordinate system, and X_{Mtr}, Y_{Mtr}, and Z_{Mtr} represent an X-axis, a Y-axis, and a Z-axis of the mechanical arm motion platform dynamic coordinate system, respectively.

In an embodiment, the motion platform can be a Stewart platform, or the other platform, which is not limited in the present disclosure.

Taking the mechanical arm controlling the held ultrasound probe through the motion platform as an example, a determination of the plane equations corresponding to the first ultrasound section and the second ultrasound section in the same probe coordinate system in the above embodiments can be achieved through steps S1021 to S1023, which are described as follows:

Step S1021: Calculating a transformation matrix from a mechanical coordinate system to the probe coordinate system according to a transformation relationship between coordinate systems.

The transformation relationship between coordinate systems here includes a transformation matrix from the mechanical coordinate system to the static coordinate system, a transformation matrix from the static coordinate system to the dynamic coordinate system, and a transformation matrix from the dynamic coordinate system to the probe coordinate system. Wherein, the static coordinate system and the dynamic coordinate system are established based on the mechanical arm. Specifically, the static coordinate system and the dynamic coordinate system can be reference coordinate systems required to be established according to the kinematic analysis of the mechanical arm.

Taking the mechanical arm controlling the held ultrasound probe through the motion platform as an example, the static coordinate system and the dynamic coordinate system are established based on the motion platform on the mechanical arm. The static coordinate system can also be referred to as the mechanical arm motion platform static coordinate system, and the dynamic coordinate system can be referred to as the mechanical arm motion platform dynamic coordinate system.

In an embodiment of the present application, the mechanical coordinate system can also be known as a global coordinate system or a world coordinate system, which is a coordinate system established at a base of the mechanical arm according to rules of the world coordinate system. It is usually set at a center of the base of the mechanical arm and positioned directly below a first joint of multi-joint arm, enabling a transformation relationship between the mechanical coordinate system and the first joint to be as simple as possible. Assuming that the mechanical coordinate system is denoted as F₀-X₀Y₀Z₀, an origin point F₀ is fixedly connected to the base of the mechanical arm, the Z₀-axis points to a movable joint from F₀, the Y₀-axis points to the mechanical arm from the base F₀, and a direction of x₀-axis conforms to a right-hand coordinate system.

In one embodiment, the mechanical arm may include the first mechanical arm and the second mechanical arm, then a first static coordinate system and a first dynamic coordinate system can be established based on the first mechanical arm; a second static coordinate system and a second dynamic coordinate system can be established based on the second mechanical arm. Here, the first mechanical arm holds the first ultrasound probe defined in the previous embodiment, and the second mechanical arm holds the second ultrasound probe defined in the previous embodiment.

Taking both the first mechanical arm and the second mechanical arm controlling the held ultrasound probe through the motion platform as an example, the first static coordinate system can also be referred to as a first mechanical arm motion platform static coordinate system, and the second static coordinate system can also be referred to as a second mechanical arm motion platform static coordinate system. The first dynamic coordinate system can also be referred to as a first mechanical arm motion platform dynamic coordinate system, and the second dynamic coordinate system can also be referred to as a second mechanical arm motion platform dynamic coordinate system corresponding to the second mechanical arm.

In one embodiment, the probe coordinate system includes the first probe coordinate system corresponding to the first ultrasound probe and the second probe coordinate system corresponding to the second ultrasound probe.

In one embodiment, an origin point of the first probe coordinate system coincides with an end point of the first ultrasound probe, and an origin point of the second probe coordinate system coincides with an end point of the second ultrasound probe. Z-axis of both probe coordinate systems respectively coincides with Z-axis of the dynamic platform dynamic coordinate system of the respective mechanical arm. Further, in an initial state, the X-axis and the Y-axis of the probe coordinate system are respectively parallel to the X-axis and the Y-axis of the motion platform dynamic coordinate system. A rotation amount θₘ of a rotating motor set between the ultrasound probe and the dynamic platform is regarded as a motion of the ultrasound probe detection plane relative to the probe coordinate system.

First, according to a definition of the coordinate system, a transformation matrix T_{trans_m_det} from the motion platform dynamic coordinate system to the probe coordinate system can be obtained:

$${T}_{trans\_m \_det} = [ \begin{matrix}cos ( {θ}_{m} ) & - sin ( {θ}_{m} ) & 0 & 0 \\ sin ( {θ}_{m} ) & cos ( {θ}_{m} ) & 0 & 0 \\ 0 & 0 & 1 & moz \\ 0 & 0 & 0 & 1\end{matrix} ]$$

Wherein, moz is a distance from the origin point of the motion platform dynamic coordinate system to the end point of the ultrasound probe, i.e., the origin point of the probe coordinate system, which is a fixed constant. In addition, it should be noted that the first ultrasound section is in the plane formed by the x-axis and z-axis of the first probe coordinate system, and the second ultrasound section is in the plane formed by the x-axis and z-axis of the second probe coordinate system.

According to the rule of Denavit-Hartenberg (DH), a homogeneous transformation from a coordinate of a (i−1)ₜₕ joint to a coordinate of an iₜₕ joint is constructed as a sequence with two rotations and two transformations, which can be represented by a matrix as follows:

$${i}_{i - 1}^{} T = [ \begin{matrix}cos {θ}_{i} & - sin {θ}_{i} & 0 & {a}_{i - 1} \\ sin {θ}_{i} \times cos {α}_{i - 1} & cos {θ}_{i} \times cos {α}_{i - 1} & - sin {α}_{i - 1} & - {d}_{i} \times sin {α}_{i - 1} \\ sin {α}_{i - 1} \times sin {θ}_{i} & sin {α}_{i - 1} \times cos {θ}_{i} & cos {α}_{i - 1} & {d}_{i} \times cos {α}_{i - 1} \\ 0 & 0 & 0 & 1\end{matrix} ]$$

Wherein, i=2, 3, 4 . . . , n, and n is the total number of rotational and translation joints of the mechanical arm. A transformation matrix ₙ⁰T a 0ₜₕ mechanical coordinate system to a nₜₕ joint coordinate system can be expressed as:

$${}_{n}^{0} T = {}_{1}^{0} T \cdot {}_{2}^{1} T … {}_{n - 1}^{n - 2} T \cdot {}_{n}^{n - 1} T$$

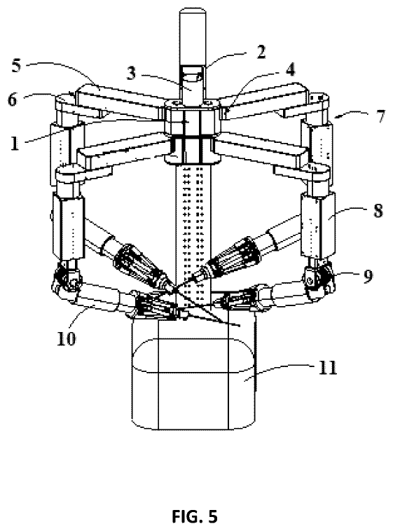

According to a configuration of a medical robot shown in FIG. 5 (which includes, in sequence, a translation joint 1, a rotational joint 2, a translation joint 3, a rotational joint 4, a rotational joint 5, a translation joint 6, a rotational joint 7, a translation joint 8, a rotational joint 9, and a translation joint 10. In addition, the medical robot also includes a base 11 fixedly connected to the translation joint 1), the transformation matrix T_{trans_mach_s} from the mechanical coordinate system to the motion platform static coordinate system is:

T_{trans_mach_s}=₁₀⁰T

During a master-slave control process, the transformation matrix T_{trans_s_m} from the motion platform static coordinate system to the motion platform dynamic coordinate system can be calculated in real time as:

$${T}_{t rans\_s \_m} = [ \begin{matrix}cos ( {λ}_{y} ) & 0 & sin ( {λ}_{y} ) & {m}_{ox} \\ sin ( {λ}_{y} ) sin ( {λ}_{x} ) & cos ( {λ}_{x} ) & - sin ( {λ}_{x} ) cos ( {λ}_{y} ) & {m}_{oy} \\ - cos ( {λ}_{x} ) sin ( {λ}_{y} ) ) & sin ( {λ}_{y} ) & cos ( {λ}_{y} ) cos ( {λ}_{x} ) & {m}_{oz} \\ 0 & 0 & 0 & 1\end{matrix} ]$$

Wherein, mₒₓ, m_{oy}, and m_{oz} are coordinates of the origin point of the motion platform dynamic coordinate system in the motion platform static coordinate system, and λₓ and λ_{y} are Euler angles, which can be understood as a current pose of a motion platform after it rotates by an angle λₓ around its own X_{M}-axis from an initial position, and then rotates by an angle λ_{y} around its own Y_{M}-axis.

In an embodiment of the present application, the calculation of the transformation matrix from the mechanical coordinate system to the probe coordinate system according to the transformation relationship of the coordinate systems can be achieved through the following steps S1 to S4, described as follows:

Step S1: obtaining a first transformation matrix A by left-multiplying the transformation matrix T_{trans_s1_m1} from the first static coordinate system to the first dynamic coordinate system by the transformation matrix T_{trans_m1_det1} from the first dynamic coordinate system to the first probe coordinate system.

Taking the first mechanical arm controlling the held ultrasound probe through the motion platform as an example, the first static coordinate system can also be referred to as the first mechanical arm motion platform static coordinate system, and the first dynamic coordinate system can also be referred to as the first mechanical arm motion platform dynamic coordinate system.

Assume the transformation matrix from the first static coordinate system to the first dynamic coordinate system is T_{trans_s1_m1}, the transformation matrix from the first dynamic coordinate system to the first probe coordinate system is T_{trans_m1_det1}, and the first transformation matrix is A, then A=T_{trans_s1_m1}·T_{trans_m1_det1}.

Step S2: obtaining a transformation matrix from the mechanical coordinate system to the first probe coordinate system by left-multiplying the transformation matrix T_{trans_mach_s1} from the mechanical coordinate system to the first static coordinate system by the first transformation matrix A.

Assume the transformation matrix from the mechanical coordinate system to the first static coordinate system is T_{trans_mach_s1}, the transformation matrix from the mechanical coordinate system to the first probe coordinate system is T_{trans_mach_det1}, then T_{trans_mach_det1}=T_{trans_mach_s1}·A=T_{trans_mach_s1}·T_{trans_s1_m1}·T_{trans_m1_det1}.

Step S3: obtaining a second transformation matrix by left-multiplying the transformation matrix from the second static coordinate system to the second dynamic coordinate system by the transformation matrix from the second dynamic coordinate system to the second probe coordinate system.

Taking the second mechanical arm controlling the held ultrasound probe through the motion platform as an example, the second static coordinate system can also be referred to as the second mechanical arm motion platform static coordinate system, and the second dynamic coordinate system can also be referred to as the second mechanical arm motion platform dynamic coordinate system.

Assume the transformation matrix from the second static coordinate system to the second dynamic coordinate system is T_{tran_s2_m2}, the transformation matrix from the second dynamic coordinate system to the second probe coordinate system is T_{trans_m2_det2}, and the second transformation matrix is defined as B, then B=T_{tran_s2_m2}·T_{trans_m2_det2}.

Step S4: obtaining a transformation matrix from the mechanical coordinate system to the second probe coordinate system by left-multiplying the transformation matrix T_{trans_mach_s2} from the mechanical coordinate system to the second static coordinate system by the second transformation matrix B.

Assume the transformation matrix from the mechanical coordinate system to the second static coordinate system is T_{trans_mach_s2}, the transformation matrix from the mechanical coordinate system to the second probe coordinate system is T_{trans_mach_det2}, and then T_{trans_mach_det2}=T_{trans_mach_s2}·B=T_{trans_mach_s2}·T_{trans_s2_m2}·T_{trans_m2_det2}.

It should be noted that the above method of calculating the transformation matrix from the mechanical coordinate system to the probe coordinate system takes the dynamic platform as an example. In practice, if the mechanical arm is not equipped with the motion platform, other types of reference coordinate systems can also be established on respective mechanical arms. Thus, for each mechanical arm, the transformation matrix from the mechanical coordinate system to the probe coordinate system can be determined, according to a transformation matrix 1 from the mechanical coordinate system to the reference coordinate system on the mechanical arm and a transformation matrix 2 from the reference coordinate system to the probe coordinate system. The transformation matrix is equal to the transformation matrix 1·the transformation matrix 2, for example.

Step S1022: calculating a transformation matrix between the first probe coordinate system and the second probe coordinate system according to the transformation matrix from the mechanical coordinate system to the probe coordinate system.

In one embodiment, the transformation matrix from the first probe coordinate system to the second probe coordinate system can be obtained by left-multiplying an inverse matrix of a transformation matrix from the first probe coordinate system to the mechanical coordinate system by a transformation matrix from the second probe coordinate system to the mechanical coordinate system.

For example, assume the inverse matrix of the transformation matrix from the mechanical coordinate system to the first probe coordinate system is T_{trans_mach_det1}⁻¹, and the transformation matrix from the mechanical coordinate system to the second probe coordinate system is T_{trans_mach_det2}, then T_{trans_det1_det2}=T_{trans_mach_det1}⁻¹·T_{trans_mach_det2}.

Step S1023: Calculating a plane equation corresponding to the first ultrasound section and a plane equation corresponding to the second ultrasound section in the same probe coordinate system, according to a normal vector of the first ultrasound section, a normal vector of the second ultrasound section and the transformation matrix between the first probe coordinate system and second probe coordinate system.

Based on a relationship between the second ultrasound section and the second probe coordinate system, it is known that the normal vector n₂ of the second ultrasound section is n₂=(0 1, 0), and the end point of the second ultrasound probe, i.e., the origin point of the second probe coordinate system, is in the second ultrasound section.

As an embodiment of the present application, if the same probe coordinate system described in the above embodiment is the first probe coordinate system, then calculating the plane equation corresponding to the first ultrasound section and the plane equation corresponding to the second ultrasound section in the same probe coordinate system, according to the normal vector of the first ultrasound section, the normal vector of the second ultrasound section and the transformation matrix between the first probe coordinate system and second probe coordinate system can be performed the following steps S′1 to S′3:

Step S′1: According to the transformation matrix from the first probe coordinate system to second probe coordinate system, obtaining a normal vector n__{1_2} of the second ultrasound section in the first probe coordinate system by transforming the normal vector n__{2_2} of the second ultrasound section in the second probe coordinate system to the first probe coordinate system, and obtaining coordinates C__{1_2} of a second specified point in the first probe coordinate system by transforming coordinates C__{2_2} of the second specified point in the second probe coordinate system to the first probe coordinate system. The second specified point is within the second ultrasound section.

In an embodiment, the second specified point may be the end point of the second ultrasound probe. According to the method of establishing the second ultrasound probe coordinate system in the embodiment, the end point is an origin point of the second ultrasound probe coordinate system (0, 0, 0), which can be represented as homogeneous coordinates [0 0 0 1].

Specifically, a homogeneous matrix n__{2_2} of the normal vector of the second ultrasound section in the second probe coordinate system is n__{2_2}=[0 1 0 1]^{T}, then the normal vector of the second ultrasound section in the first probe coordinate system is n__{1_2}=T_{trans_det1_det2}·n__{2_2}, and the coordinates of the second specified point C__{1_2} in the first probe coordinate system is C__{1_2}=T_{trans_det1_det2}·[0 0 0 1]^{T}. Obviously, the normal vector n__{1_2} of the second ultrasound section and the coordinates of the specified point C__{1_2} in the first probe coordinate system are both transposed matrixes of a 4×1 order or 4-dimensional vector, which meet a rule of matrix operation.

Step S′2: obtaining the plane equation corresponding to the second ultrasound section in the first probe coordinate system by using a point-normal form, according to components of the normal vector n__{1_2} of the x-axis, y-axis, and z-axis of the first probe coordinate system and the coordinates of the second specified point C__{1_2} of x-axis, y-axis, and z-axis of the second probe coordinate system in the first probe coordinate system.

Assume the 1st to 3rd components of the normal vector n__{1_2} of the second ultrasound section in the first probe coordinate system in the direction of the x-axis, y-axis, and z-axis in the first probe coordinate system are n__{1_2} (1), n__{1_2} (2), and n__{1_2} (3) respectively; coordinates of the 1st to 3rd elements of the second specified point C__{1_2} in the first probe coordinate system in the direction of the x-axis, y-axis, and z-axis of the second probe coordinate system are C__{1_2} (1), C__{1_2} (2), and C__{1_2} (3) respectively, then, obtaining the plane equation corresponding to the second ultrasound section in the first probe coordinate system by using a point-normal form is given as:

$${n}_{\_ 1 \_ 2} ( 1 ) \cdot ( x - {C}_{\_ 1 \_ 2} ( 1 ) ) + {n}_{\_ 1 \_ 2} ( 2 ) \cdot ( y - {C}_{\_ 1 \_ 2} ( 2 ) ) + {n}_{\_ 1 \_ 2} ( 3 ) \cdot ( z - {C}_{\_ 1 \_ 2} ( 3 ) ) = 0 \qquad ( 1 )$$

Step S′3: obtaining the plane equation corresponding to the first ultrasound section in the first probe coordinate system by using the point-normal form, according to the normal vector n₁ of the first ultrasound section in the first probe coordinate system and coordinates C₁ of the first specified point within the first ultrasound section in the first probe coordinate system.

In one embodiment, the first specified point may be the end point of the first ultrasound probe. According to the method of establishing the first probe coordinate system in the embodiment, the end point can be the origin point (0, 0, 0) of the first probe coordinate system.

The normal vector of the first ultrasound section in the first probe coordinate system is n₁ and n₁=(0, 1, 0), and the coordinates of the first specified point within the first ultrasound section in the first probe coordinate system is C₁ and C₁=(0, 0, 0). Then, the plane equation corresponding to the first ultrasound section in the first probe coordinate system obtained by using the point-normal form is:

$$[ y = 0 ] \qquad ( 2 )$$

As for the jointly solving of the plane equations corresponding to the first ultrasound section and the second ultrasound section within the same probe coordinate system, to obtain the intersecting line of the first ultrasound section and the second ultrasound section in the above mentioned embodiments, it may be specifically, jointly solving the plane equation corresponding to the second ultrasound section in the first probe coordinate system and the plane equation corresponding to the first ultrasound section in the first probe coordinate system, obtaining a straight line corresponding to the linear equation as the puncture travel path of the puncture needle. It can be seen from the aforementioned embodiment that the plane equation corresponding to the second ultrasound section in the first probe coordinate system is n__{1_2}(1)·(x−C__{1_2}(1))+n__{1_2}(2)·(y−C__{1_2}(2))+n__{1_2}(3)·(z−C__{1_2}(3))=0, and the plane equation corresponding to the first ultrasound section in the first probe coordinate system is y=0. Therefore, by jointly solving the above two plane equations, the line equation as follows is obtained: n__{1_2} (1)·(x−C__{1_2} (1))+n__{1_2} (3)·(z−C__{1_2} (3))−n__{1_2} (2)·C__{1_2} (2)=0.

It should be noted that, as another embodiment of the present application, the jointly solving of the plane equations corresponding to the first ultrasound section and the second ultrasound section within the same probe coordinate system, to obtain the intersecting line of the first ultrasound section and the second ultrasound section, can also be achieved by jointly solving the plane equation corresponding to the first ultrasound section in the second probe coordinate system and the plane equation corresponding to the second ultrasound section in the second probe coordinate system and obtaining a straight line corresponding to the linear equation as the puncture travel path of the puncture needle. The solution method of the plane equation corresponding to the first ultrasound section in the second probe coordinate system, is similar to the solution method of the plane equation corresponding to the second ultrasound section in the first probe coordinate system in the previous embodiment. The solution method of the plane equation corresponding to the second ultrasound section in the second probe coordinate system is similar to the solution method of the plane equation corresponding to the first ultrasound section in the first probe coordinate system in the previous embodiment. Please refer to the related descriptions in the previous embodiment for details, which will not be repeated here.

It should be noted that in the above description, the ultrasound probe is illustrated as the first ultrasound probe and the second ultrasound probe, and the mechanical arm is illustrated as the first mechanical arm and the second mechanical arm, it is not intended to limit the number of the mechanical arms and the ultrasound probes.

If the first mechanical arm and the second mechanical arm are two different mechanical arms, the two mechanical arms may respectively hold two different ultrasound probes. The first ultrasound probe held by the first mechanical arm is used to collect the first ultrasound section. The second ultrasound probe held by the second mechanical arm is used to collect the second ultrasound section.

If the first mechanical arm and the second mechanical arm are the same mechanical arm, the mechanical arm holds a single ultrasound probe and collects two different ultrasound sections at different times. Among them, the above mentioned first mechanical arm is in fact a first position of the mechanical arm when collecting the first ultrasound section, and the above mentioned second mechanical arm is in fact a second position of the mechanical arm when collecting the second ultrasound section.

In step S103, determining a puncture travel path according to the intersecting line, when the first ultrasound section and/or the second ultrasound section meet(s) a preset condition. The puncture travel path is a travel path along which the puncture needle performs puncturing.

In one embodiment, the puncture needle may be held by a third mechanical arm. The third mechanical arm may control the puncture needle through a motion platform, or can also control the puncture needle through other ways, which is not limited in the embodiment.

During movements of the first ultrasound probe and/or the second ultrasound probe, when the acquired first ultrasound section and/or the second ultrasound section meet(s) the preset condition, the movement of the first ultrasound probe and/or the second ultrasound probe can be stopped. At this time, the intersecting line of the two ultrasound sections is the straight line where the puncture travel path is located.

The first ultrasound section and/or the second ultrasound section that meet(s) the preset condition can be determined by the operator of the puncture surgery according to experience. In principle, as long as a current ultrasound section satisfies the operator of the puncture surgery, the current ultrasound section can be regarded as the first ultrasound section and/or the second ultrasound section. Then, the operator of the puncture surgery can issue commands to stop moving the mechanical arm. If the first ultrasound section and/or the second ultrasound section do not meet(s) the preset condition, then it returns to the step of controlling the ultrasound probe held by the mechanical arm for movement. That is, the ultrasound probe held by the mechanical arm is re-adjusted for movement.

In one embodiment, if two mechanical arms controls two ultrasound probes separately, according to the commands of the operator, one of the ultrasound probes can be fixed to a suitable position first, and then the other ultrasound probe can be controlled to move. Then, according to the intersecting line displayed in the two ultrasound sections, the operator can determine whether the two ultrasound sections meet the preset condition. Of course, if no ultrasound section that meet(s) the preset condition can be obtained by moving the other ultrasound probe, the ultrasound probe fixed in the suitable position can be moved again according to the operator's commands. Alternatively, according to the operator's commands, both ultrasound probes can be moved simultaneously, and then the intersecting line displayed in the two ultrasound sections can be used for the operator to determine whether the two ultrasound sections meet the preset condition.

As previously mentioned, since the puncture travel path actually coincides with the intersecting line of the first ultrasound section and the second ultrasound section, by fine-tuning at least one ultrasound probe held by at least one mechanical arm, the intersecting line of the first ultrasound section and the second ultrasound section can be synchronously displayed on the first ultrasound image and the second ultrasound image. Therefore, the puncture travel path can be synchronously displayed on the first ultrasound image and the second ultrasound image, enabling the operator to observe the entire puncture needle in real time, accurately locate the needle tip, and monitor the puncture process throughout, thereby improving the safety of the surgery.

In related technologies, three-dimensional ultrasound images can be used to guide the operator to determine the puncture path. However, three-dimensional ultrasound images have a low quality, a large modeling delay, with safety hazards. Alternatively, in related technologies, single two-dimensional ultrasound image is usually used to guide the puncture, but two-dimensional ultrasound can only view planar images, making it difficult to view the position of the needle tip or even the entire needle in planar images, leading to difficulty in operation and easily losing the needle position, resulting in safety hazards. According to the embodiments of the present application, on the one hand, the use of the two-dimensional ultrasound images is simpler and less delay than the three-dimensional ultrasonic modeling algorithm. On the other hand, using the intersecting line of two ultrasound images to determine the puncture path enables the spatial position of the puncture needle to be located in the subsequent puncture process, allowing the operator to locate the entire puncture needle in real time during puncture, avoiding a problem of unable to view the entire puncture needle position in a single two-dimensional ultrasound image.

In addition, in the embodiment of the present application, on the one hand, since the mechanical arm can be precisely controlled by a computer program, there is no jitter during the movement of the mechanical arm holding the ultrasound probe and the puncture needle, making it extremely stable and reliable compared to manual operation. On the other hand, the generated puncture travel path accurately locates the spatial position of the puncture needle, allowing the puncture needle to be accurately positioned along the puncture travel path when puncturing to the target point. In a third aspect, by synchronously displaying the puncture travel path on the first ultrasound image and the second ultrasound image, the operator can observe the entire puncture needle in real time, accurately locate the position of the needle tip, and monitor the entire puncture process, thereby improving the safety of the surgery.

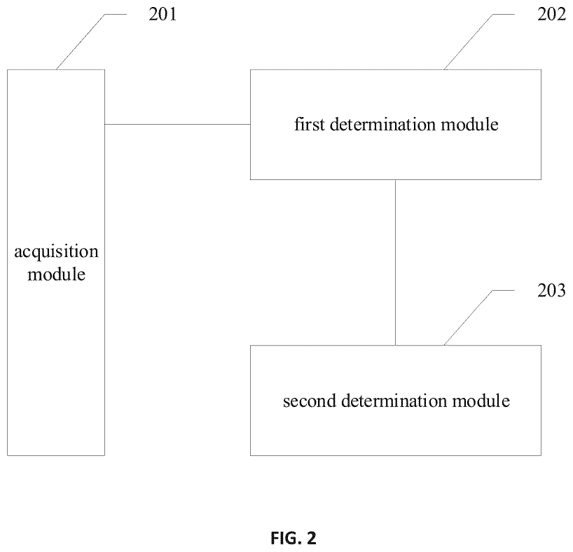

Referring to FIG. 2, it shows a structural schematic diagram of a path determination device provided in an embodiment of the present application. For convenience of illustration, only parts related to the embodiment of the present application are shown. The device can be a computer terminal or a software module configured in the computer terminal. As shown in FIG. 2, the device includes: an acquisition module 201, a first determination module 202, and a second determination module 203, detailed as follows:

The acquisition module 201, is configured to obtain a first ultrasound section and a second ultrasound section collected by at least one ultrasound probe.

The first determination module 202 is configured to respectively determine an intersecting line of the first ultrasound section and the second ultrasound section in the first ultrasound section and the second ultrasound section, respectively.

The second determination module 203 is configured to determine a puncture travel path according to the intersecting line when the first ultrasound section and/or the second ultrasound section meet(s) a preset condition. The puncture travel path is a travel path along which the puncture needle performs puncturing.

Furthermore, if the first ultrasound section and the second ultrasound section do not meet the preset condition, the first manipulation module 201 continues to control the at least one of a first ultrasound probe held by a first mechanical arm and a second ultrasound probe held by a second mechanical arm to move.

Additionally, the first ultrasound section is collected by the first ultrasound probe, and the second ultrasound section is collected by the second ultrasound probe. The first ultrasound probe and the second ultrasound probe may be the same ultrasound probe or different ultrasound probes. The first determination module 203 is specifically used to determine plane equations corresponding to the first ultrasound section and the second ultrasound section in a same probe coordinate system, and obtain the intersecting line of the first ultrasound section and the second ultrasound section by jointly solving the plane equation corresponding to the first ultrasound section and the plane equation corresponding to the second ultrasound section in the same probe coordinate system. Wherein, the same probe coordinate system may be the first probe coordinate system or the second probe coordinate system. The first probe coordinate system is established according to the first ultrasound probe, and the second probe coordinate system is established according to the second ultrasound probe.

Furthermore, the above step of determining plane equations corresponding to the first ultrasound section and the second ultrasound section in a same probe coordinate system may include: calculating a transformation matrix of the mechanical coordinate system to the probe coordinate system according to a transformation relationship between coordinate systems; calculating a transformation matrix between the first probe coordinate system and the second probe coordinate system according to the transformation matrix of the mechanical coordinate system to the probe coordinate system; calculating plane equations corresponding to the first ultrasound section and the second ultrasound section in the same probe coordinate system according to a normal vector of the first ultrasound section, a normal vector of the second ultrasound section and the transformation matrix between the first probe coordinate system and second probe coordinate system. Wherein, the transformation relationship between coordinate systems include a transformation matrix from the mechanical coordinate system to a static coordinate system, a transformation matrix from the static coordinate system to a dynamic coordinate system, and a transformation matrix from the dynamic coordinate system to the probe coordinate system. The static coordinate system and the dynamic coordinate system are established according to the mechanical arm.

Furthermore, the above-mentioned first ultrasound probe is held by a first mechanical arm, and the second ultrasound probe is held by a second mechanical arm. The static coordinate system includes a first static coordinate system and a second static coordinate system. The dynamic coordinate system includes a first dynamic coordinate system and a second dynamic coordinate system. The above step of calculating a transformation matrix of the mechanical coordinate system to the probe coordinate system, according to a transformation relationship between coordinate systems may include: obtaining a first transformation matrix A by left-multiplying the transformation matrix T_{trans_s1_m1} from the first static coordinate system to the first dynamic coordinate system by the transformation matrix T_{trans_m1_det1} from the first dynamic coordinate system to the first probe coordinate system; obtaining a transformation matrix from the mechanical coordinate system to the first probe coordinate system by left-multiplying the transformation matrix T_{trans_mach_s1} from the mechanical coordinate system to the first static coordinate system by the first transformation matrix A; obtaining a second transformation matrix B by left-multiplying the transformation matrix T_{tran_s2_m2} from the second static coordinate system to the second mechanical arm motion platform dynamic coordinate system by the transformation matrix T_{trans_m2_det2} from the second dynamic coordinate system to the second probe coordinate system; and obtaining a transformation matrix from the mechanical coordinate system to the second probe coordinate system by left-multiplying the transformation matrix T_{trans_mach_s2} from the mechanical coordinate system to the second static coordinate system by the second transformation matrix B.

Furthermore, the above-mentioned step of calculating a transformation matrix between the first probe coordinate system and the second probe coordinate system according to the transformation matrix of the mechanical coordinate system to the probe coordinate system may include: obtaining the transformation matrix between the first probe coordinate system and the second probe coordinate system by left-multiplying an inverse matrix of a transformation matrix from the first probe coordinate system to the mechanical coordinate system by a transformation matrix from the second probe coordinate system to the mechanical coordinate system.

Furthermore, the above mentioned step of calculating plane equations corresponding to the first ultrasound section and the second ultrasound section in the same probe coordinate system according to a normal vector of the first ultrasound section, a normal vector of the second ultrasound section and the transformation matrix between the first probe coordinate system and second probe coordinate system may include: according to the transformation matrix from the first probe coordinate system to second probe coordinate system, obtaining a normal vector n__{1_2} of the second ultrasound section in the first probe coordinate system by transforming the normal vector n__{2_2} of the second ultrasound section in the second probe coordinate system to the first probe coordinate system, and obtaining coordinates C__{1_2} of a second specified point in the first probe coordinate system by transforming coordinates C__{2_2} of the second specified point in the second probe coordinate system to the first probe coordinate system. The second specified point is within the second ultrasound section. The plane equation corresponding to the second ultrasound section in the first probe coordinate system is obtained by using a point-normal form, according to components of the normal vector n__{1_2} of the x-axis, y-axis, and z-axis of the first probe coordinate system and the coordinates of the second specified point C__{1_2} of x-axis, y-axis, and z-axis in the first probe coordinate system. The plane equation corresponding to the first ultrasound section in the first probe coordinate system is obtained by using the point-normal form, according to the normal vector n₁ of the first ultrasound section in the first probe coordinate system and coordinates C₁ of a first specified point within the first ultrasound section in the first probe coordinate system.

Furthermore, the second specified point may be an origin point of the second probe coordinate system, and the first specified point may be an origin point of the first probe coordinate system.

The specific processes for the above each module to achieve their respective functions can be referred to relevant contents in the embodiment shown in FIG. 1-a, which will not be repeated here.

In the embodiment of the present application, on the one hand, since the mechanical arm can be precisely controlled by a computer program, there is no jitter during the movement of the mechanical arm holding the ultrasound probe and the puncture needle, making it extremely stable and reliable compared to manual operation. On the other hand, the generated puncture travel path accurately locates the spatial position of the puncture needle, allowing the puncture needle to be accurately positioned along the puncture travel path when puncturing to the target point. In a third aspect, by synchronously displaying the puncture travel path on the first ultrasound image and the second ultrasound image, the operator can observe the entire puncture needle in real time, accurately locate the position of the needle tip, and monitor the entire puncture process, thereby improving the safety of the surgery.

An embodiment of the present application provides a medical robot, which includes at least one probe mechanical arm, a puncture mechanical arm. The probe mechanical arm holds an ultrasound probe, and the puncture mechanical arm holds a puncture needle. The medical robot also includes a control component. The control component is configured to control an ultrasound probe held by the probe mechanical arm to move and obtain a first ultrasound section and a second ultrasound section collected by the ultrasound probe; determine an intersecting line of the first ultrasound section and the second ultrasound section respectively in the first ultrasound section and the second ultrasound section; determine a puncture travel path according to the intersecting line; and control the puncture needle held by the puncture mechanical arm to perform the puncture along the puncture travel path when the first ultrasound section and/or the second ultrasound section meet(s) the preset condition.

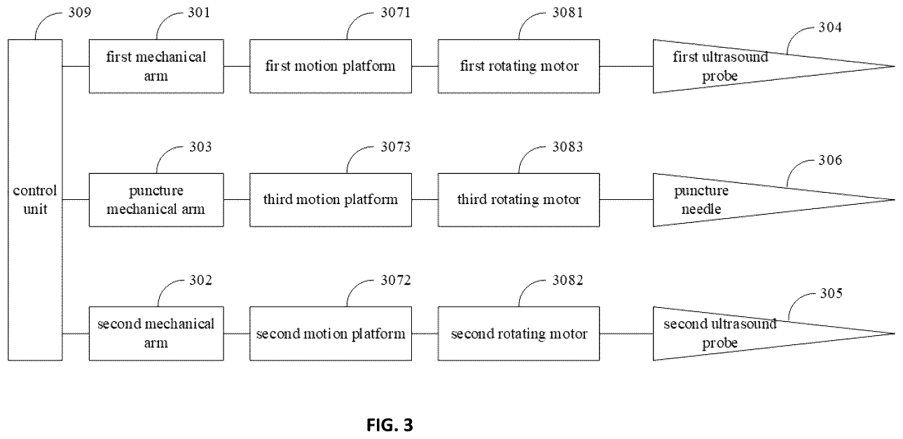

Refer to FIG. 3, shows a structural schematic diagram of a medical robot provided in an embodiment of the present application.

As shown in FIG. 3, the number of probe mechanical arms is at least two. The probe mechanical arms include a first mechanical arm and a second mechanical arm. The first mechanical arm holds a first ultrasound probe, and the second mechanical arm holds a second ultrasound probe. The first ultrasound probe is used to collect a first ultrasound section, and the second ultrasound probe is used to collect a second ultrasound section.

The medical robot includes a first mechanical arm 301, a second mechanical arm 302, a puncture mechanical arm 303, a first ultrasound probe 304 held by the first mechanical arm 301, a second ultrasound probe 305 held by the second mechanical arm 302, a puncture needle 306 held by the puncture mechanical arm 303, and a control unit 309.

The control unit 309 is configured to control at least one of the first ultrasound probe 304 held by the first mechanical arm 301 and the second ultrasound probe 305 held by the second mechanical arm 302 to move; obtain a first ultrasound section collected by the first ultrasound probe 304 and a second ultrasound section collected by the second ultrasound probe 305; determine an intersecting line of the first ultrasound section and the second ultrasound section in each of the first ultrasound section and the second ultrasound section; determine a puncture travel path according to the intersecting line; and control the puncture needle held by the puncture mechanical arm 303 to perform a puncture along the puncture travel path when the first ultrasound section and/or the second ultrasound section meet(s) a preset condition.

In one embodiment, the medical robot further includes a first motion platform, a second motion platform, a third motion platform, a first rotating motor connected to the first motion platform, a second rotating motor connected to the second motion platform, and a third rotating motor connected to the third motion platform.

The first mechanical arm 301 is connected to a first motion platform 3071, and the first motion platform 3071 is connected to the first rotating motor 3081. The first rotating motor 3081 holds and controls a movement of the first ultrasound probe 304. The second mechanical arm 302 is connected to a second motion platform 3072, and the second motion platform 3072 is connected to the second rotating motor 3082. The second rotating motor 3082 holds and controls a movement of the second ultrasound probe 30. The puncture mechanical arm 303 is connected to a third motion platform 3073, and the third motion platform 3073 is connected to the third rotating motor 3083. The third rotating motor 3083 holds and controls a movement of the puncture needle 306.

In one embodiment, the first motion platform 3071 includes a first static platform and a first dynamic platform (not shown in the figures), wherein the first static platform is connected to the first mechanical arm 301, and the first dynamic platform 3071 is connected to the first rotating motor 3081. The second motion platform 3072 includes a second static platform and a second dynamic platform (not shown in the figures), wherein the second static platform is connected to the second mechanical arm 302, and the second dynamic platform 3072 is connected to the second rotating motor 3082. The third motion platform 3073 includes a third static platform and a third dynamic platform (not shown in the figures), wherein the third static platform is connected to the puncture mechanical arm 303, and the third dynamic platform 3073 is connected to the third rotating motor 3083.

It should be noted that FIG. 3 illustrates an example where the first mechanical arm and the second mechanical arm are different mechanical arms and separately hold two ultrasound probes. In practice, the medical robot can also use a same mechanical arm (e.g., the probe mechanical arm) to hold a same ultrasound probe and acquire the first ultrasound section and the second ultrasound section at different times, which will be described as follows.

In one embodiment, the number of the probe mechanical arms is at least one, and the probe mechanical arm includes the first mechanical arm. The first mechanical arm holds a first ultrasound probe, which is used to acquire the first ultrasound section and the second ultrasound section.

The medical robot can include a first motion platform, a third motion platform, a first rotating motor connected to the first motion platform, and a third rotating motor connected to the third motion platform.

The first motion platform includes a first static platform and a first dynamic platform. The first static platform is connected to the first mechanical arm, and the first dynamic platform is connected to the first rotating motor.

The third motion platform includes a third static platform and a third dynamic platform. The third static platform is connected to the puncture mechanical arm, and the third dynamic platform is connected to the third rotating motor.

The first rotating motor holds the first ultrasound probe.

The third rotating motor holds the puncture needle.

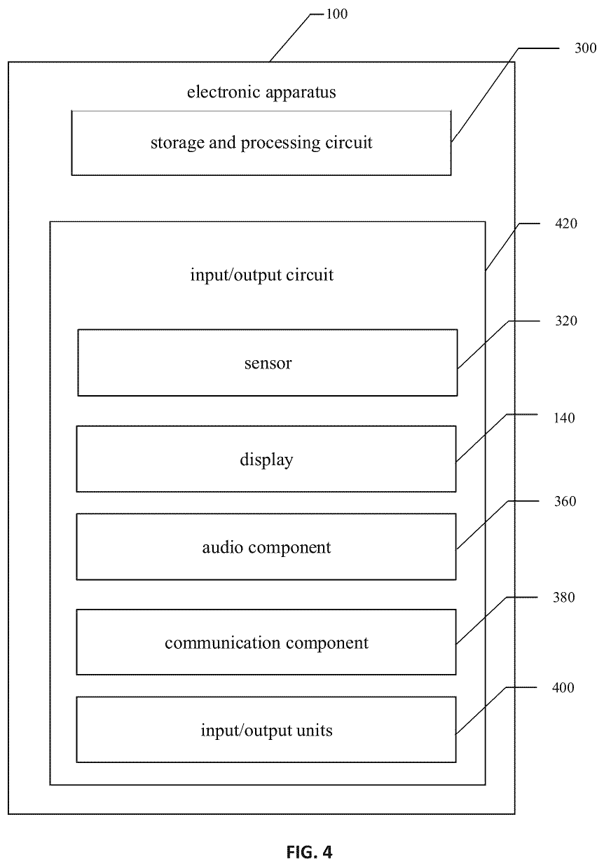

Referring to FIG. 4, it shows a structural schematic diagram of an electronic apparatus provided in an embodiment of the present application.

Illustratively, the electronic apparatus can be any type of computer system device that is non-movable, movable, or portable and performs wireless or wired communication. Specifically, the electronic apparatus can be a desktop computer, a server, a mobile phone or a smart phone (for example, based on iPhone™, Android™-based phone), a portable game device (such as Nintendo DS™, PlayStation Portable™, Gameboy Advance™, iPhone™), a laptop, a personal digital assistant PDA, a portable internet device, a portable medical device, a smart camera, a music player, a data storage device, and other handheld devices, such as a watch, a headset, a pendant, an earphone, and the like. The electronic apparatus can also be other wearable devices (for example, electronic glasses, electronic clothing, electronic bracelets, electronic necklaces, and other head-mounted devices (HMD)).

As shown in FIG. 4, the electronic apparatus 100 may include a control circuit, which may include a storage and processing circuit 300. The storage and processing circuit 300 may include a memory, such as a hard disk drive memory, a non-volatile memory (e.g., flash memory or other electronically programmable limited deletion memory used to form a solid-state drive, etc.), a volatile memory (e.g., static or dynamic random access memory, etc.), and the like, which are not limited in the embodiment of the present application. A processing circuit in the storage and processing circuit 300 can be used to control operations of the electronic apparatus 100. The processing circuit can be implemented based on one or more microprocessors, micro-controllers, digital signal processors, baseband processors, power management units, audio codec chips, application-specific integrated circuits, display driver integrated circuits, and the like.

The storage and processing circuit 300 can be used to run software in the electronic apparatus 100, such as internet browsing applications, Voice over Internet Protocol (VOIP) phone call applications, email applications, media playback applications, operating system functions, and the like. These software can be used to perform some control operations, for example, image capture based on a camera, ambient light measurement based on an ambient light sensor, proximity sensor measurement based on a proximity sensor, information display functions implemented based on status indicators such as light-emitting diodes, touch event detection based on touch sensors, functions associated with displaying information on a plurality of (e.g., layered) displays, operations associated with performing wireless communication functions, operations associated with collecting and generating audio signals, control operations associated with collecting and processing button press event data, and other functions in the electronic apparatus 100. The embodiment of the present application is not limited.

Furthermore, the memory stores executable program codes. A processor coupled to the memory calls the executable program codes stored in the memory to perform the path determination method described in the aforementioned embodiments.

The executable program codes include various modules in the path determination device described in the embodiment shown in FIG. 2, such as: a first control module 201, a second control module 202, a generation module 203, a display module 204, and a third control module 205, and the like. The specific process of realizing respective functions of the above modules can refer to the relevant descriptions of FIG. 1-a, which will not be repeated here.

The electronic apparatus 100 can also include an input/output circuit 420. The input/output circuit 420 can be used to enable the electronic apparatus 100 to achieve data input and data output, i.e., to allow the electronic apparatus 100 to receive data from external devices and also to allow the electronic apparatus 100 to output data from the electronic apparatus 100 to external devices. The input/output circuit 420 can further include a sensor 320. The sensor 320 may include an ambient light sensor, a proximity sensor based on light and capacitance, a touch sensor (for example, a touch sensor based on optical touch and/or capacitive touch, wherein the touch sensor can be a part of a touch display screen or can be used independently as a touch sensor structure), an acceleration sensor, and other sensors etc.

The input/output circuit 420 may also include one or more displays, such as a display 140. The display 140 may include a combination of one or several of a liquid crystal display, an organic light-emitting diode display, an electronic ink display, a plasma display, and a display using other display technologies. The display 140 can include a touch sensor array (i.e., the display 140 can be a touch display screen). The touch sensor can be a capacitive touch sensor formed by a transparent touch sensor electrode (such as an indium tin oxide (ITO) electrode) array, or can be a touch sensor formed using other touch technologies, such as acoustic touch, pressure-sensitive touch, resistive touch, optical touch, etc. The embodiment of the present application is not limited.

The electronic apparatus 100 may also include an audio component 360. The audio component 360 can be used to provide audio input and output functions for the electronic apparatus 100. The audio component 360 in the electronic apparatus 100 can include speakers, microphones, buzzers, tone generators, and other components used to generate and detect sound.

A communication circuit 380 can be used to provide the electronic apparatus 100 with an ability to communicate with external devices. The communication circuit 380 can include analog and digital input/output interface circuits, and wireless communication circuits based on radio frequency signals and/or optical signals. A wireless communication circuit in the communication circuit 380 can include a radio frequency transceiver circuit, a power amplifier circuit, a low-noise amplifier, switches, filters, and antennas. For example, the wireless communication circuit in the communication circuit 380 can include a circuit that supports Near Field Communication (NFC) by transmitting and receiving near-field coupled electromagnetic signals. For instance, the communication circuit 380 can include an NFC antenna and an NFC transceiver. The communication circuit 380 can also include a cellular phone transceiver and an antenna, a wireless local area network transceiver circuit and antenna, and the like.

The electronic apparatus 100 may further include a battery, a power management circuit, and other input/output units 400. The input/output units 400 can include a button, a joystick, a click wheel, a scroll wheel, a touchpad, a keypad, a keyboard, a camera, a light-emitting diode, and other status indicators etc.

Users can input commands through the input/output circuit 420 to control the operation of the electronic apparatus 100, and can use the output data of the input/output circuit 420 to receive status information and other outputs from the electronic apparatus 100.

Furthermore, the embodiments of the present application also provide a non-transitory computer-readable storage medium, which can be configured in the servers of the above-mentioned embodiments. The non-transitory computer-readable storage medium stores computer programs, which implements the path determination method described in the above embodiments when executed by a processor.

In the above embodiments, the descriptions of each embodiment focus on different aspects, and parts not described or recorded in detail in a certain embodiment can refer to the relevant descriptions of other embodiments.

The skilled in the art can appreciate that the modules/units and algorithm steps described in combination with the examples disclosed in the embodiments of the present application can be implemented in electronic hardware, or a combination of computer software and electronic hardware. Whether these functions are executed in a hardware or software manner depends on specific applications and design constraints of the technical solutions. Professionals in the field can use different methods to implement the described functions for each specific application, but such implementation should not be considered to exceed the scope of the present invention.

In the embodiments provided in the present application, it should be understood that the disclosed devices/terminals and methods can be implemented in other ways. For example, the described device/terminal embodiments are merely illustrative, and the division of modules or units is only a logical function division. In actual implementation, there can be other division methods. For example, a plurality of units or components can be combined or integrated into another system, or some features can be ignored or not executed. Another point is that the coupling or direct coupling or communication connection displayed or discussed can be an indirect coupling or communication connection through some interfaces, devices, or units, which can be electrical, mechanical, or other forms.

The units described as separate components may be or may be not physically separated. The components displayed as units can be or can be not physical units, i.e., they may be located in one place or may be distributed across a plurality of network units. Part or all of the units can be selected according to actual needs to achieve the purpose of the embodiments of the present application.

In addition, the functional units in each embodiment of the present invention can be integrated into a processing unit, or can exist physically separately, or can integrate two or more units into a single unit. The integrated unit can be implemented in a form of hardware or as a software functional unit.

If the integrated unit is implemented in the form of a software functional unit and sold or used as an independent product, it can be stored in a computer-readable storage medium. Based on this understanding, the present invention implements all or part of the processes in the above-mentioned embodiment methods, which can also be completed by instructing relevant hardware through a computer programs. The computer programs can be stored in a computer-readable storage medium, and when executed by a processor, the computer program can realize the steps of each method embodiment described above. Among them, the computer program includes computer program code, which can be in the form of source code, object code, executable files, or some intermediate forms. Computer-readable media can include: any entity or device capable of carrying computer program code, recording media, USB flash drives, mobile hard drives, magnetic disks, optical discs, computer memory, read-only memory (ROM), random access memory (RAM), electrical carrier signals, telecommunications signals, and software distribution media, etc. It should be noted that the content contained in the computer-readable medium can be appropriately added or deleted according to the requirements of legislation and patent practice in the jurisdiction, for example, in some jurisdictions, according to legislation and patent practice, computer-readable media does not include electrical carrier signals and telecommunications signals.

The above embodiments are only used to illustrate the technical solutions of the present invention, rather than to limit them. Although the present invention has been described in detail with reference to the foregoing embodiments, those skilled in the art should understand that they can still modify the technical solutions recorded in the foregoing embodiments, or replace some technical features with equivalent ones; and these modifications or replacements do not make the essence of the corresponding technical solutions depart from the spirit and scope of the technical solutions of the embodiments of the present invention, and should be included within the protection scope of the present invention.

1. A path determination method, comprising steps of:
obtaining a first ultrasound section and a second ultrasound section collected by at least one ultrasound probe;
determining an intersecting line of the first ultrasound section and the second ultrasound section; and
determining a puncture travel path according to the intersecting line when receiving a stop command triggered based on the first ultrasound section and/or the second ultrasound section.

2. The path determination method according to claim 1, wherein the step of obtaining a first ultrasound section and a second ultrasound section collected by at least one ultrasound probe comprises:
obtaining the first ultrasound section collected by a first ultrasound probe at a first position and obtaining the second ultrasound section collected by a second ultrasound probe at a second position.

3. The path determination method according to claim 2, wherein the step of determining an intersecting line of the first ultrasound section and the second ultrasound section comprises:
determining a plane equation corresponding to the first ultrasound section and a plane equation corresponding to the second ultrasound section in a same probe coordinate system; and
obtaining the intersecting line of the first ultrasound section and the second ultrasound section based on the plane equation corresponding to the first ultrasound section and the plane equation corresponding to the second ultrasound section.

4. The path determination method according to claim 3, wherein the same probe coordinate system is either a first probe coordinate system or a second probe coordinate system, the first probe coordinate system being established based on the first probe coordinate system, and the second probe coordinate system being established based on the second probe coordinate system.

5. The path determination method according to claim 4, wherein the step of determining a plane equation corresponding to the first ultrasound section and a plane equation corresponding to the second ultrasound section in a same probe coordinate system comprises:
calculating a transformation matrix from a mechanical coordinate system to the probe coordinate system according to a transformation relationship between coordinate systems;
calculating a transformation matrix between the first probe coordinate system and the second probe coordinate system according to the transformation matrix from the mechanical coordinate system to the probe coordinate system; and
determining the plane equation corresponding to the first ultrasound section and the plane equation corresponding to the second ultrasound section in the same probe coordinate system according to a normal vector of the first ultrasound section, a normal vector of the second ultrasound section and the transformation matrix between the first probe coordinate system and second probe coordinate system.

6. The path determination method according to claim 5, wherein the transformation relationship between coordinate systems comprises a transformation matrix from the mechanical coordinate system to a static coordinate system, a transformation matrix from the static coordinate system to a dynamic coordinate system, and a transformation matrix from the dynamic coordinate system to the probe coordinate system, and wherein the static coordinate system and the dynamic coordinate system are established according to a mechanical arm.

7. The path determination method according to claim 6, wherein the first ultrasound probe is held by a first mechanical arm, and the second ultrasound probe is held by a second mechanical arm,
wherein the static coordinate system comprises a first static coordinate system and a second static coordinate system, the dynamic coordinate system comprises a first dynamic coordinate system and a second dynamic coordinate system, the first static coordinate system and the first dynamic coordinate system coordinate system are established according to the first mechanical arm, and the second static coordinate system and the second dynamic coordinate system coordinate system are established according to the second mechanical arm; and
wherein the step of calculating a transformation matrix from the mechanical coordinate system to the probe coordinate system according to a transformation relationship between coordinate systems comprises:
obtaining a first transformation matrix A by left-multiplying a transformation matrix T_{trans_s1_m1} from the first static coordinate system to the first dynamic coordinate system by a transformation matrix T_{trans_m1_det1} from the first dynamic coordinate system to the first probe coordinate system;
obtaining a transformation matrix from the mechanical coordinate system to the first probe coordinate system by left-multiplying a transformation matrix T_{trans_mach_s1} from the mechanical coordinate system to the first static coordinate system by the first transformation matrix A;
obtaining a second transformation matrix B by left-multiplying a transformation matrix T_{tran_s2_m2} from the second static coordinate system to the second dynamic coordinate system by a transformation matrix T_{trans_m2_det2} from the second dynamic coordinate system to the second probe coordinate system; and
obtaining a transformation matrix from the mechanical coordinate system to the second probe coordinate system by left-multiplying a transformation matrix T_{trans_mach_s2} from the mechanical coordinate system to the second static coordinate system by the second transformation matrix B.

8. The path determination method according to claim 7, wherein the step of calculating a transformation matrix between the first probe coordinate system and the second probe coordinate system according to the transformation matrix of the mechanical coordinate system to the probe coordinate system comprises:
obtaining the transformation matrix between the first probe coordinate system and the second probe coordinate system by left-multiplying an inverse matrix of the transformation matrix from the first probe coordinate system to the mechanical coordinate system by the transformation matrix from the second probe coordinate system to the mechanical coordinate system.

9. The path determination method according to claim 7, wherein the same probe coordinate system is the first probe coordinate system, wherein the step of determining a plane equation corresponding to the first ultrasound section and a plane equation corresponding to the second ultrasound section in the same probe coordinate system according to a normal vector of the first ultrasound section, a normal vector of the second ultrasound section and the transformation matrix between the first probe coordinate system and second probe coordinate system comprises:
according to the transformation matrix between the first probe coordinate system and second probe coordinate system, obtaining a normal vector n__{1_2} of the second ultrasound section in the first probe coordinate system by transforming a normal vector n__{2_2} of the second ultrasound section in the second probe coordinate system to the first probe coordinate system;
obtaining coordinates C__{1_2} of a second specified point in the first probe coordinate system by transforming coordinates C__{2_2} of the second specified point in the second probe coordinate system to the first probe coordinate system, the second specified point is within the second ultrasound section;
obtaining the plane equation corresponding to the second ultrasound section in the first probe coordinate system by using a point-normal form according to components of the normal vector n__{1_2} of the x-axis, y-axis, and z-axis of the first probe coordinate system and the coordinates C__{1_2} of the second specified point of x-axis, y-axis, and z-axis in the first probe coordinate system; and
obtaining the plane equation corresponding to the first ultrasound section in the first probe coordinate system by using the point-normal form according to the normal vector n₁ of the first ultrasound section in the first probe coordinate system and coordinates C₁ of a first specified point within the first ultrasound section in the first probe coordinate system.

10. The path determination method according to claim 9, wherein
the second specified point is an origin point of the second probe coordinate system; and
the first specified point is an origin point of the first probe coordinate system.

11. The path determination method according to claim 1, further comprising:
synchronously displaying the intersecting line of the first ultrasound section and the second ultrasound section on a first ultrasound image corresponding to the first ultrasound section and a second ultrasound image corresponding to the second ultrasound section.

12. A path determination device, comprising:
acquisition module, configured to obtain a first ultrasound section and a second ultrasound section collected by at least one ultrasound probe;
a first determination module, configured to determine an intersecting line of the first ultrasound section and the second ultrasound section; and
a second determination module, configured to determine a puncture travel path according to the intersecting line when receiving a stop command triggered based on the first ultrasound section and/or the second ultrasound section.

13. A medical robot, comprising at least one probe mechanical arm and a puncture mechanical arm, the at least one probe mechanical arm holding an ultrasound probe, and the puncture mechanical arm holding a puncture needle, the medical robot further comprising a control component,
wherein the control component is configured to control the ultrasound probe held by the probe mechanical arm to move and obtain a first ultrasound section and a second ultrasound section collected by the ultrasound probe, determine an intersecting line of the first ultrasound section and the second ultrasound section, and determine a puncture travel path according to the intersecting line when receiving a stop command triggered based on the first ultrasound section and/or the second ultrasound section, and wherein the puncture travel path is used to indicate the puncture needle held by the puncture mechanical arm during puncture.

14. The medical robot according to claim 13, wherein the at least one probe mechanical arm comprises a first mechanical arm and a second mechanical arm, the first mechanical arm holding a first ultrasound probe, and the second mechanical arm holding a second ultrasound probe, the first ultrasound probe being used to collect the first ultrasound section, and the second ultrasound probe being used to collect the second ultrasound section.

15. The medical robot according to claim 14, further comprising a first motion platform, a second motion platform, a third motion platform, a first rotating motor connected to the first motion platform, a second rotating motor connected to the second motion platform, and a third rotating motor connected to the third motion platform:
wherein the first motion platform comprises a first static platform and a first dynamic platform, the first static platform is connected to the first mechanical arm, and the first dynamic platform is connected to the first rotating motor;
wherein the second motion platform comprises a second static platform and a second dynamic platform, the second static platform is connected to the second mechanical arm, and the second dynamic platform is connected to the second rotating motor;
wherein the third motion platform comprises a third static platform and a third dynamic platform, the third static platform is connected to the puncture mechanical arm, and the third dynamic platform is connected to the third rotating motor;
wherein the first rotating motor holds the first ultrasound probe;
wherein the second rotating motor holds the second ultrasound probe; and
wherein the third rotating motor holds the puncture needle.

16. The medical robot according to claim 13, wherein the probe mechanical arm comprises a first mechanical arm, the first mechanical arm holding a first ultrasound probe, and being used to collect the first ultrasound section and the second ultrasound section.

17. The medical robot according to claim 16, further comprising: a first motion platform, a third motion platform, a first rotating motor connected to the first motion platform, and a third rotating motor connected to the third motion platform,
wherein the first motion platform comprises a first static platform and a first dynamic platform, the first static platform is connected to the first mechanical arm, and the first dynamic platform is connected to the first rotating motor;
wherein the third motion platform comprises a third static platform and a third dynamic platform, the third static platform is connected to the puncture mechanical arm, and the third dynamic platform is connected to the third rotating motor;
wherein the first rotating motor holds the first ultrasound probe; and
wherein the third rotating motor holds the puncture needle.

18. An electronic apparatus, comprising a memory and a processor,
wherein the memory stores executable program codes; and
wherein the processor is coupled with the memory and configured to call the executable program codes stored in the memory, and execute a path determination method according to claim 1.

19. A computer-readable storage medium having stored thereon commands that, when executed by a processor, causes to perform a path determination method according to claim 1.
